(19) United States
(12) Patent Application Publication
(10) Pub. No.: US 2007/0185502 A1
(43) Pub. Date: Aug. 9, 2007

(54) Medical electrode delivery tool

(76) Inventor: Karel Smits, Limburg (NL)

Correspondence Address: MEDTRONIC, INC., 710 MEDTRONIC PARKWAY NE, MINNEAPOLIS, MN, 55432-9924, US

(21) Appl. No.: 11/321,390

(22) Filed: Dec. 29, 2005

(51) Int. Cl. A61B 19/00

(52) U.S. Cl. 606/129

(57) ABSTRACT

A medical electrode delivery tool includes a distal portion coupled to an elongate shaft. The distal portion includes a generally ellipsoidal segment. The ellipsoidal segment includes a concave surface forming a cavity having an opening facing in a direction away from a longitudinal axis of the shaft and a convex surface overlying the concave surface and tapering to a distal end of the ellipsoidal segment.

### BACKGROUND

Certain embodiments in the present disclosure pertain to medical electrode delivery and more particularly to tools for delivering medical electrodes.

Positioning of one or more medical electrodes at implant sites within a body of a patient, via minimally invasive techniques, may be difficult without the assistance of a delivery tool. Some such tools are constructed to help an implanting physician push electrodes to the implant site, while other types of these tools are constructed to set up a pathway to the implant site through which the implanting physician may pass the electrodes without encountering anatomical obstructions.

In certain instances, a patient suffering from bradycardia, tachyarrhythmia and/or heart failure will benefit from electrical stimulation pacing and/or defibrillation electrodes implanted on an epicardial surface of the patient's heart. Minimally invasive methods for accessing the epicardial surface, which is enclosed within a pericardial sac, have recently been developed; these methods provide for piercing through the pericardial sac in order to access the epicardial surface; an example of one such method is described in commonly assigned U.S. Pat. No. 6,837,848. These methods may be used by way of a mini-thoracotomy or in conjunction with a trocar, canula or catheter that has been passed, via a percutaneous incision, through an interstitial space between the patient's ribs or by a sub-xiphoid approach; those skilled in the art are familiar with these techniques.

Once access to the epicardial surface is established, the implanting physician may insert a medical electrical lead, including an appropriate electrode configuration suited to the patient's need, into the pericardial space. The physician will almost always need to maneuver the electrode-bearing portion of the lead within the space in order to implant the epicardial electrode(s) at an appropriate location and in a way to provide effective and stable electrical stimulation therapy; an electrode delivery tool can greatly facilitate this process.

### SUMMARY

Certain embodiments of the present invention pertain to a medical electrode delivery tool including a distal portion coupled to an elongate shaft; the distal portion includes a generally ellipsoidal segment, which includes a concave surface forming a cavity having an opening facing in a direction away from a longitudinal axis of the shaft and a convex surface overlying the concave surface and tapering to a distal end of the ellipsoidal segment. The elongate shaft may include a pre-formed curve formed in a distal length thereof and/or one or more longitudinally extending lumens in fluid communication with the cavity. Certain embodiments of the present invention may further include a holding element coupled to the concave surface and/or at least one longitudinally extending slot passing through the concave and convex surfaces of the ellipsoidal segment.

It should be noted that, although most embodiments of the present invention are described herein in the context of epicardial pacing and/or defibrillation, the invention is not so limited. Those skilled in the art will appreciate that there are numerous regions of the body wherein electrical stimulation may be beneficial and which present similar obstacles to electrode delivery that may be overcome by embodiments of the present invention; examples of these regions include, but are not limited to, regions of the nervous system and the gastrointestinal system.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a schematic view of an exemplary delivery tool positioned for medical electrode delivery to an epicardial surface of a heart, according to some embodiments of the present invention.

FIG. 2A is top plan view of the tool shown in FIG. 1, according to some embodiments of the present invention.

FIG. 2B is a side plan view of the tool shown in FIG. 2A.

FIG. 2C is a section view through section line A-A of FIG. 2A.

FIG. 2D is a section view through section line B-B of FIG. 2A, according to one embodiment of the present invention.

FIGS. 3A-B are section views through section line B-B of FIG. 2A, according to alternate embodiments of the present invention.

FIG. 4A is a top plan view of a portion of a delivery tool, according to alternate embodiments of the present invention, directed for insertion through a trocar.

FIG. 4B is a section view through section line G-G of FIG. 4A.

FIG. 5A is a plan view a delivery tool according to further embodiments of the present invention.

FIG. 5B is a schematic section view of a heart and the tool shown in FIG. 4A positioned about the epicardial surface of the heart.

FIG. 6A is a side plan view including a cut-away section of a system including a delivery tool according to further embodiments of the present invention.

FIG. 6B is a top plan view of a distal portion of the tool shown in FIG. 6A, according to one embodiment of the present invention.

FIG. 6C is a top plan view of a distal portion of the tool shown in FIG. 6A, according to another embodiment of the present invention.

FIG. 6D is an enlarged section view of a portion of the tool shown in FIG. 6A.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention.

FIG. 1 is a schematic view of an exemplary delivery tool 100 positioned for medical electrode delivery to an epicardial surface of a heart 1, according to some embodiments of the present invention. FIG. 1 illustrates delivery tool 100 including an elongate shaft 11 and a distal portion 13 coupled to shaft 11; distal portion 13 is shown inserted between an epicardial surface of heart 1 and a pericardial sac 4 surrounding heart 1 through a pericardial incision 10. According to certain embodiments of the present invention, a shape of distal portion 13 facilitates insertion thereof between the pericardium and epicardium. Distal portion 13 is described in greater detail below.

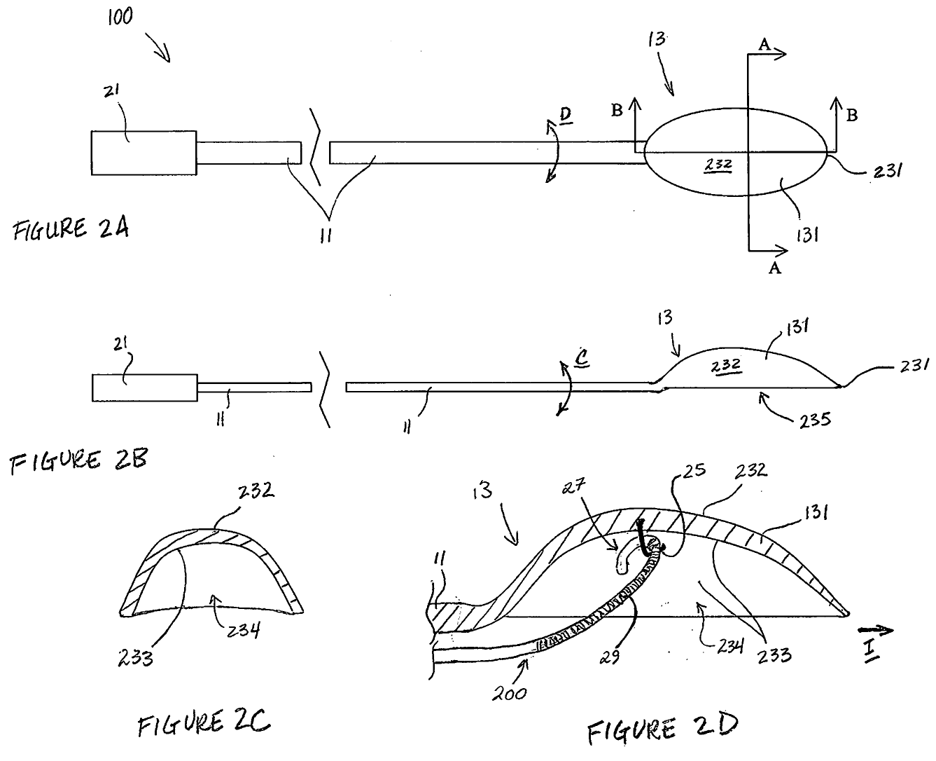

FIGS. 2A-B are top and side plan views of delivery tool 100; and FIGS. 2C-D are section views through section lines A-A and B-B, respectively, shown in FIG. 2A. FIGS. 2A-D illustrate distal portion 13 including a generally ellipsoidal segment 131; ellipsoidal segment 131 is shown including a concave surface 233, forming a cavity 234 having an opening 235 facing in a direction away from a longitudinal axis of shaft 11, and a convex surface 232 overlying concave surface 233 and tapering to a distal end 231. According to the illustrated embodiments, an operator or implanting physician, grasping shaft 11 at or near a handle 21 thereof, would maneuver distal portion 13 to an electrode implant site, for example as illustrated in FIG. 1, such that opening 235 of ellipsoidal segment faces toward the implant site, for example on the epicardial surface of heart 1, and convex surface 232 faces away from the implant site, for example contacting pericardium 4.

Distal portion 13, shaft 11 and handle 21 may be formed of any suitable biocompatible material or a combination of these materials known to those skilled in the art, for example medical grade metal and/or polymer materials. Distal portion 13 and handle 21 may be coupled to shaft 11 by any suitable means known to those skilled in the art, examples of which include but are not limited to adhesive bonding, welding and press fitting. According to some embodiments, tool 100 as a whole is injection molded from a polymer having a relatively high modulus of elasticity, yet being sufficiently elastic and not prone to brittle fracture, for example 75D durometer polyurethane or high density polyethylene or polyamide. Alternately, handle 21 and/or distal portion 13, may be insert molded onto shaft 11, which may have been formed by molding or an extrusion process. According to some embodiments, shaft 11 may be wholly or partially formed from a metal having suitable elastic and elasto-plastic properties, examples of which include, but are not limited to, titanium alloys, Ni-Ti super-elastic alloys and stainless steel.

According to some embodiments of the present invention, shaft 11 has stiffness properties making shaft relatively flexible to bend in a first direction, for example, according to double-headed arrow C, which is out of plane from cavity opening 235 as shown in FIG. 2B, and relatively stiff to resist bending in a second direction which is approximately normal to the first direction, for example, represented by double-headed arrow D shown in FIG. 2A. Such stiffness properties may facilitate maneuvering of tool 100 by allowing cavity opening 235 to move up and down for tracking over a contour of a surface (i.e. per arrow C), while providing lateral control for placing distal portion 13. A shaft cross-sectional geometry having a greater thickness in one direction than in another, for example as seen in comparing FIG. 2A to FIG. 2B, may lend such stiffness properties to shaft 11; alternately, or additionally, elements built into shaft 11, according to construction details known to those skilled in the art, can provide the desired stiffness properties.

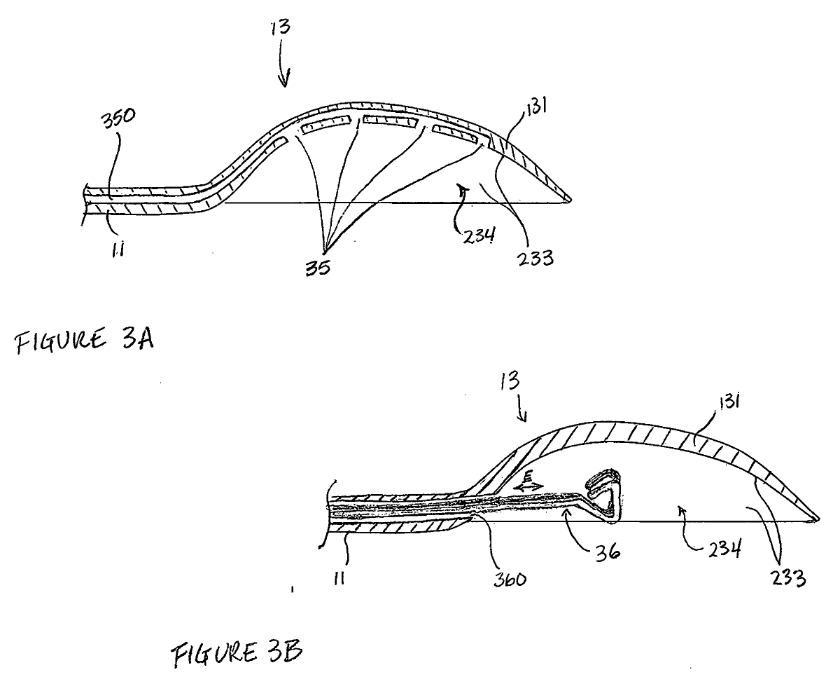

With reference to FIGS. 2A-B it may be appreciated how the tapering of convex surface 232 to distal end 231 can facilitate insertion of distal portion 13 between opposing layers of anatomical structures, for example between the pericardium and the epicardium, and that cavity 234, formed by concave surface 233 of ellipsoid segment 131, can provide a space in which at least one medical electrode may be inserted for implantation; the electrode may be carried to the implant site, within cavity 234, by distal portion 13, or may be inserted into cavity 234 for implantation after distal portion 13 is positioned at the implant site. Some embodiments of the present invention wherein a medical electrode is carried within cavity 234 are described in conjunction with FIGS. 2D and 3A-B.

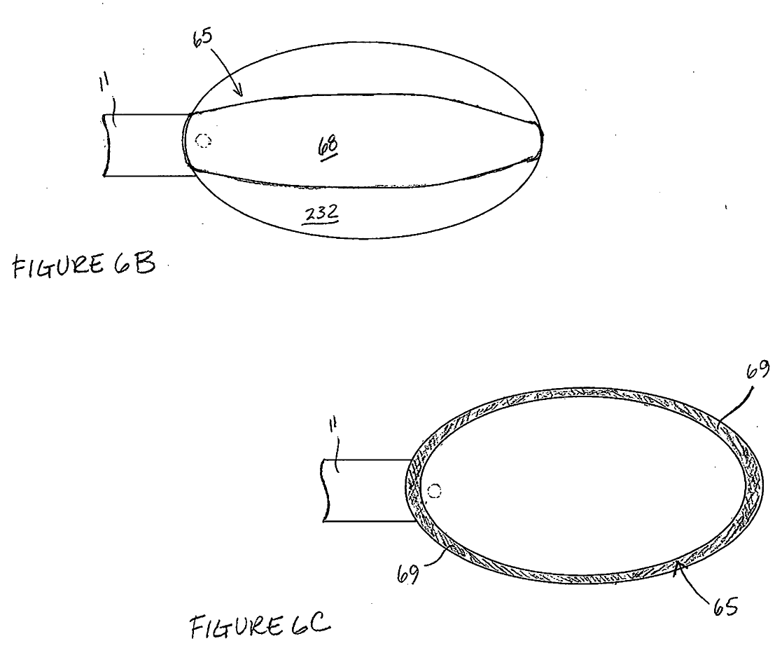

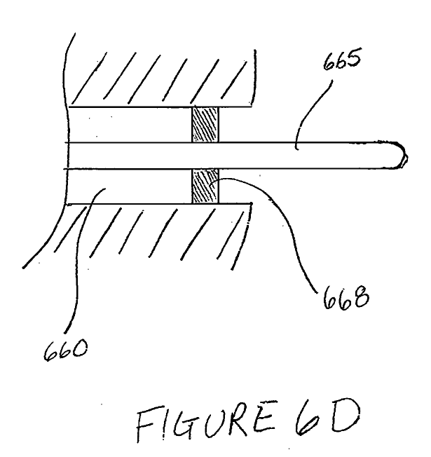

FIG. 2D illustrates distal portion 13 further including a holding element 25 formed as a hook for carrying a medical electrode 29 to an implant site. According to the illustrated embodiment, electrode 29, in the form of an elongate coil, for example a defibrillation electrode, is coupled to a distal portion of a medical electrical lead body 200 in proximity to a curved distal tip 27 thereof, which is shown held by hook 25. With further reference to FIG. 2D, it may be appreciated that hook 25 will carry electrode 29, when distal portion 13 is advanced, per arrow 1, toward an implant site. Although FIG. 2D illustrates lead body 200 extending proximally outside of shaft 11, alternate embodiments of the present invention include a shaft having a longitudinally extending lumen to accommodate medical electrical leads for delivery of the lead electrodes; one such embodiment will be described below, in conjunction with FIG. 6.

FIGS. 3A is a section view through section line B-B of FIG. 2A, according to an alternate embodiment of the present invention. FIG. 3A illustrates shaft 11 including a longitudinally extending vacuum lumen 350 that terminates in vacuum ports 35 formed in concave surface 233 of segment 131. Ports 35, via a suction force applied through lumen 350, may serve as holding elements for an electrode to carry the electrode to an implant site. Alternately or additionally ports 35, via the suction force, may help hold cavity 234 in intimate contact with an underlying surface to provide stability for an electrode implant procedure. Although FIG. 3A illustrates a plurality of vacuum ports 35, a single vacuum port is not outside the scope of the present invention.

FIG. 3B is a section view through section line B-B of FIG. 2A, according to another alternate embodiment. FIG. 3B illustrates a holding element formed by a clamping mechanism, the clamping mechanism including gripping tongs 36 extending longitudinally within lumen 360 of shaft 11. According to the illustrated embodiment, gripping tongs 36 extend from proximal ends (not shown) to terminate distally within cavity 234 where tongs 36 can releasably grip an electrode or an electrode-bearing portion of a lead. With reference to the double-headed arrow E, tongs 36 may be advanced and retracted, via pushing a pulling the proximal ends of tongs. Advancing tongs 36 distally causes tongs 36 open and release, and retracting tongs 36 proximally causes tongs to close and grip. Additionally tongs 36 may be rotated about their longitudinal axis; such rotation can be used to implant a hook or helix electrode, which are well known to those skilled in the art. Commonly assigned U.S. Pat. No. 6,010,526 describes such a mechanism and relevant parts are incorporated by reference herein.

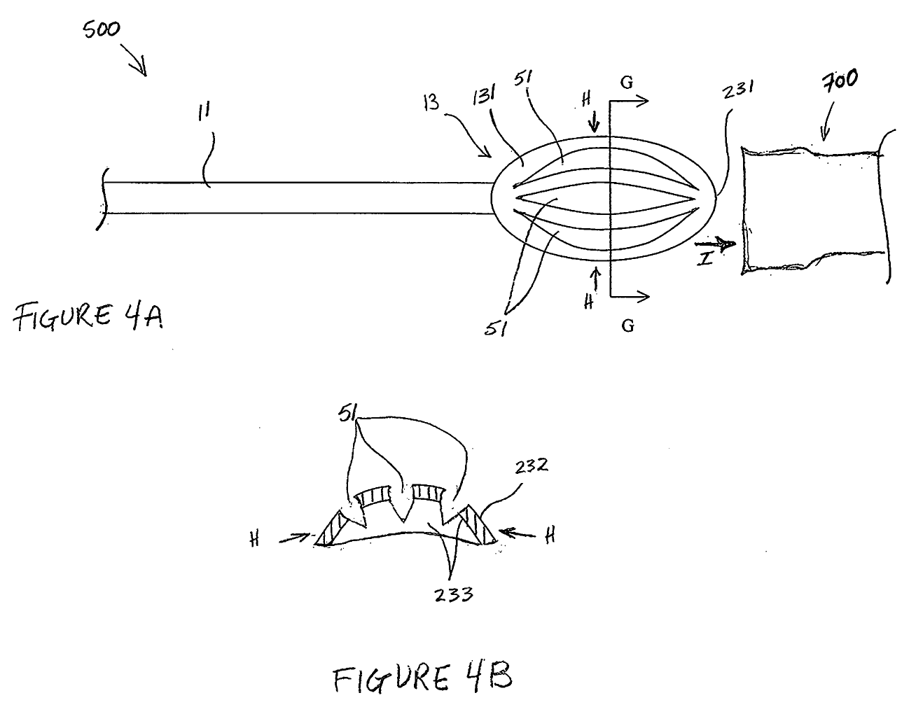

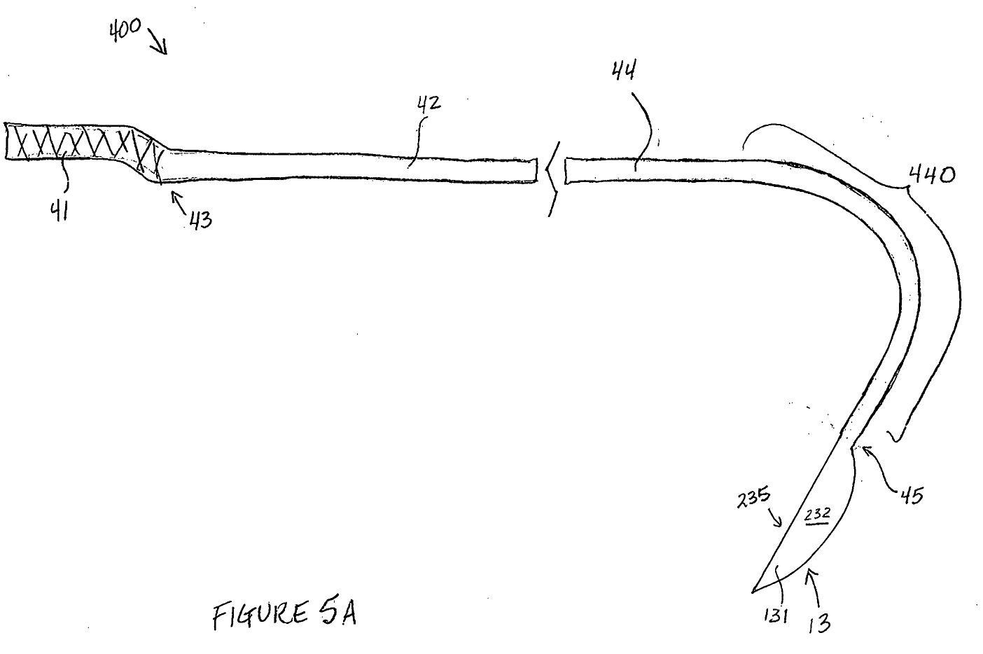

FIG. 4A is a top plan view of a portion of a delivery tool 500, according to alternate embodiments of the present invention, directed for insertion, per arrow 1, through a trocar 700. FIG. 4B is a section view through section line G-G of FIG. 4A. FIGS. 4A-B illustrate generally ellipsoidal segment 131 including longitudinally extending slots 51 passing through convex and concave surfaces 232, 233. According to the illustrated embodiment, slots 51 allow for a compression of segment 131, per arrows H, to facilitate passage of distal portion 13 through a lumen of trocar 700 into proximity with an implant site, for example, through incision 10 shown in FIG. 1. Although FIGS. 4A-B show a plurality of slots 51, segment 131 including a single slot is not outside the scope of the present invention. Furthermore any of the alternate embodiments of distal portion 13 described herein may further include one or more of this type of slot.

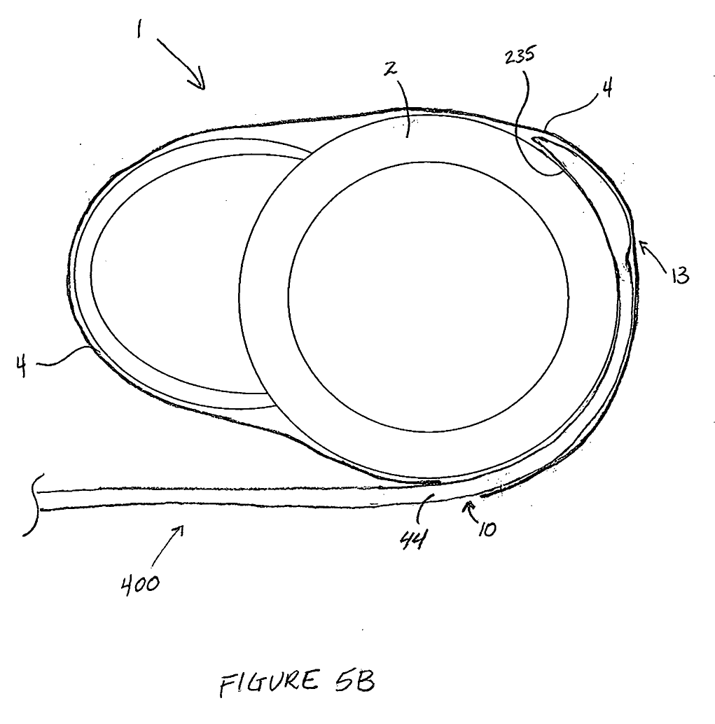

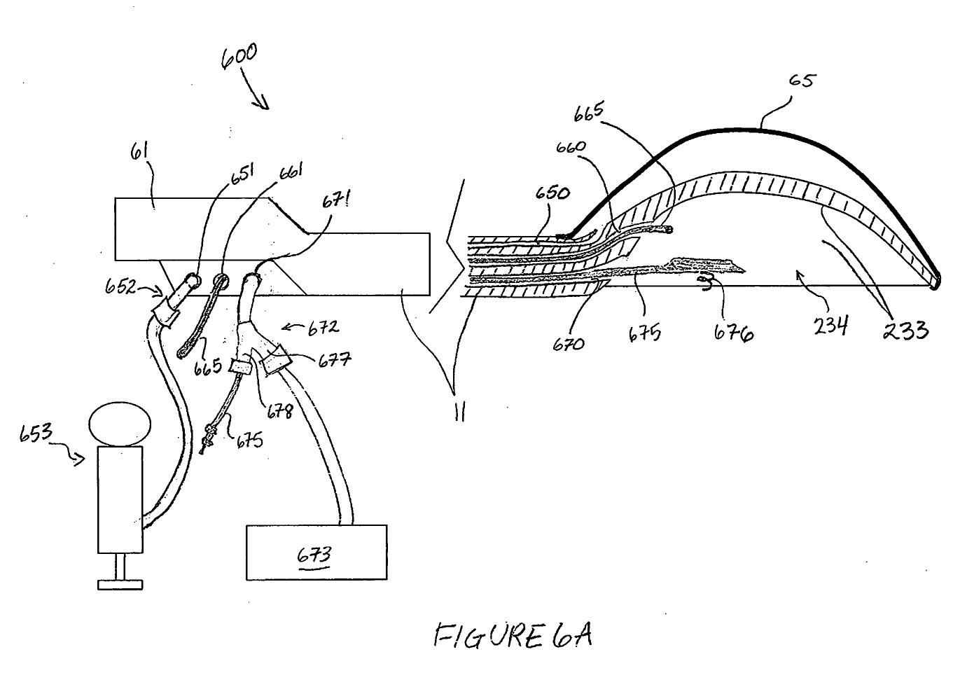

FIG. 5A is a plan view of a delivery tool 400 according to further embodiments of the present invention. FIG. 5B is a schematic section view of heart 1 where tool 400 is positioned about the epicardial surface of a left ventricle 2 of heart 1. FIG. 5A illustrates delivery tool 400 including a shaft proximal length 42, terminated at a proximal end 43 by a handle 41, and a shaft distal length 44, terminated at a distal end 45 by distal portion 13. Shaft distal length 44 is shown including a pre-formed curvature 440. Pre-formed curvature 440 may be formed according to methods known to those skilled in the art, either in a primary shaft-forming process or by a secondary heat-treating of shaft 11. According to some embodiments of the present invention, pre-formed curvature 440 has a bend radius smaller than that of a mating anatomical surface. For example, with reference to FIG. 5B, a radius of the left ventricular epicardial surface of heart 1 is greater than that of preformed curvature 440 shown in FIG. 5A, so that distal portion 13 and shaft distal length 44, having been inserted beneath pericardial sac 4, through incision 10, exert pressure against the epicardial surface rather than against pericardial sac 4 when shaft distal length 44 is stretched open by the curvature of the epicardial surface. This pressure against the epicardial surface may provide sufficient stability to implant an electrode from cavity opening 235. It should be noted that any of the features of distal portion 13, described in conjunction with the preceding figures, may be incorporated into tool 400 to form additional alternate embodiments of the present invention.

FIG. 6A is a side plan view including cut-away sections of a delivery tool 600 according to further embodiments of the present invention; and FIGS. 6B-C are top plan views of a distal portion of the tool shown in FIG. 6A, according to alternate embodiments. FIG. 6A illustrates delivery tool 600 including a distal portion 63 coupled to shaft 11 and including an inflatable member 65 extending over convex surface 232 of ellipsoidal segment 131. When inflated, as illustrated in FIG. 6A, member 65 creates a protrusion that may help to force cavity 234 of segment 131 into closer contact with an implant site surface, by pressing against an opposing surface. Member 65 may form a relatively narrow ridge-like protrusion 68 extending longitudinally over a central portion of convex surface 232, as shown in FIG. 6B, or member 65 may extend over a wider swath of surface 232, as shown in FIG. 6C. Inflatable member 65 may be formed from a sheet of material, either relatively compliant, for example silicone rubber, or relatively non-compliant, for example a cross-linked polyethylene, bonded or welded about a perimeter 69 thereof to ellipsoidal segment 131 (FIG. 6C); alternate methods for manufacturing such members or balloons on medical devices are known to those skilled in the art. According to the illustrated embodiment, lumen 650 serves as an inflation lumen for member 65 and includes a proximal access port 651 disposed in proximity to a handle 61 of shaft 11 to which a fitting 652, for example a luer lock fitting, is coupled for coupling of lumen 650 to an inflation device 653, such as is known to those skilled in the art.

FIG. 6A further illustrates shaft 11 including lumens 660 and 670. Proximal ends of tool 665 and lead 675 are shown extending proximally from proximal access ports 661 and 671, corresponding to lumens 660 and 670, respectively. According to the illustrated embodiment, lumen 660 serves as a passageway for a visualization tool 665, for example a fiber-optic scope, and lumen 670 serves as a passageway for a medical electrical lead 675; lumen 670 may further serve as a vacuum lumen and a proximal port 671 of lumen 670 is shown coupled to a y-adaptor fitting 672 for coupling of lumen 670 to a vacuum source 673, via a first leg 677 of the fitting, while allowing passage of lead 675 therethrough into lumen 670, through a second leg 678 of the fitting. Visualization tool 665, having been inserted in a proximal access port 661 of lumen 660 and positioned as shown, can assist an implanting physician or operator in navigating tool 600 to an implant site and further assist the operator in implanting a medical electrode, for example helix electrode 676 shown extending from a distal end of medical electrical lead 675. It should be noted that one or both of lumens 660 and 670 may be incorporated into the embodiments described in conjunction with any of the preceding figures to form additional alternate embodiments of the present invention.

With further reference to FIG. 6A, it should be appreciated that second leg 678 of y-adaptor fitting 672 will include an element to seal around lead 675, when vacuum is drawn through first leg 677, so that the vacuum is maintained in lumen 670 for application to cavity 234; an example of such an element is a Toughy-Borst seal known to those skilled in the art. A sealing element within lumen 660 may also be required to maintain the vacuum, and FIG. 6D is an enlarged section view of a portion of tool 600 showing a sealing element 668 disposed within a distal portion of lumen 660 according to some embodiments of the present invention. FIG. 6D illustrates element 668 sealing lumen 660 around visualization tool 665 while allowing passage of tool 665 therethrough; sealing element 668 may be a pierceable elastic membrane or a split grommet mounted within lumen 660 according to construction methods known to those skilled in the art.

In the foregoing detailed description, the invention has been described with reference to specific embodiments. However, it may be appreciated that various modifications and changes can be made without departing from the scope of the invention as set forth in the appended claims.

1. A medical electrode delivery tool, comprising:
an elongate shaft; and
a distal portion coupled to the shaft, the distal portion including:
a generally ellipsoidal segment, the ellipsoidal segment including a concave surface forming a cavity having an opening facing in a direction away from a longitudinal axis of the shaft and a convex surface overlying the concave surface and tapering to a distal end of the ellipsoidal segment; and
a holding element disposed within the cavity for holding an electrode or a portion of a lead body bearing the electrode within the cavity.

2. A delivery tool according to claim 1, wherein the elongate shaft includes at least one longitudinally extending lumen in fluid communication with the cavity of the distal portion.

3. A delivery tool according to claim 1, wherein:
the distal portion further includes an inflatable member extending over the convex surface of the ellipsoidal segment; and
the shaft includes a longitudinally extending lumen in fluid communication of the inflatable member for inflation thereof.

4. A delivery tool according to claim 1, wherein the shaft has stiffness properties making the shaft relatively flexible to bend in a first direction and relatively stiff to resisting bending in a second direction that is approximately normal to the first direction, the first direction being out of plane from the opening of the cavity.

5. A delivery tool according to claim 1, wherein:
the elongate shaft includes a longitudinally extending vacuum lumen; and
the distal portion includes at least one vacuum port in fluid communication with the vacuum lumen and the cavity.

6. A delivery tool according to claim 1, wherein the holding element comprises a hook coupled to the concave surface of the ellipsoidal segment.

7. A delivery tool according to claim 1, wherein the holding element comprises a clamping mechanism.

8. A delivery tool according to claim 1, wherein the elongate shaft includes a distal length, the distal length including a pre-formed curvature.

9. A medical electrode delivery tool comprising:
an elongate shaft including a proximal length and a distal length, the distal length including a portion having a pre-formed curvature; and
a distal portion coupled to a distal end of the distal length of the shaft, the distal portion including a generally ellipsoidal segment, the ellipsoidal segment including a concave surface forming a cavity having an opening facing in a direction away from a longitudinal axis of the shaft and a convex surface overlying the concave surface and tapering to a distal end of the ellipsoidal segment.

10. A delivery tool according to claim 9, wherein the elongate shaft includes at least one longitudinally extending lumen in fluid communication with the cavity of the distal portion.

11. A delivery tool according to claim 9, wherein:
the distal portion further includes an inflatable member extending over the convex surface of the ellipsoidal segment; and
the shaft includes a longitudinally extending lumen in fluid communication of the inflatable member for inflation thereof.

12. A delivery tool according to claim 9, wherein the shaft includes stiffness properties adjusted to make the shaft relatively flexible to bend in a first direction and relatively stiff to resisting bending in a second direction that is approximately normal to the first direction, the first direction being out of plane from the opening of the cavity.

13. A delivery tool according to claim 9, wherein:
the elongate shaft includes a longitudinally extending vacuum lumen; and
the distal portion includes at least one vacuum port in fluid communication with the vacuum lumen and the cavity.

14. A delivery tool according to claim 13, further comprising a sealing element coupled to the vacuum lumen, the sealing element allowing passage of an elongate member through the vacuum lumen while sealing around the member.

15. A delivery tool according to claim 13, wherein the shaft further includes a longitudinally extending visualization lumen in fluid communication with the cavity, the visualization lumen providing passage for an elongate visualization tool into the cavity and including a sealing member formed therein to seal about the visualization tool extending therethrough.

16. A delivery tool according to claim 9, further comprising a clamping mechanism disposed within the cavity of the ellipsoidal segment for holding the electrode or a portion of a lead body bearing the electrode within the cavity.

17. A delivery tool according to claim 9, wherein the ellipsoidal segment further includes at least one longitudinally extending slot passing through the concave and convex surfaces allowing compression of the ellipsoid segment.

18. A medical electrode delivery tool comprising:
an elongate shaft; and
a distal portion coupled to the shaft, the distal portion including a generally ellipsoidal segment, the ellipsoidal segment including a concave surface forming a cavity having an opening facing in a direction away from a longitudinal axis of the shaft, a convex surface overlying the concave surface and tapering to a distal end of the ellipsoidal segment, and at least one longitudinally extending slot passing through the concave and convex surfaces allowing compression of the ellipsoid segment.

19. A delivery tool according to claim 18, wherein the elongate shaft includes at least one longitudinally extending lumen in fluid communication with the cavity of the distal portion.

20. A delivery tool according to claim 18, wherein the shaft includes stiffness properties adapted to make the shaft relatively flexible to bend in a first direction and relatively stiff to resisting bending in a second direction that is approximately normal to the first direction, the first direction being out of plane from the opening of the cavity.
